(19) United States
(12) Patent Application Publication
(10) Pub. No.: US 2013/0074841 A1
(43) Pub. Date: Mar. 28, 2013

(54) DISPENSER FOR POWDERED COMPOSITIONS CONTAINED IN A SEPARATE PACK

(71) Applicant: Alfred Von Schuckmann, Kevelaer (DE)

(72) Inventor: Alfred Von Schuckmann, Kevelaer (DE)

(21) Appl. No.: 13/703,118

(22) PCT Filed: Jun. 7, 2011
(86) PCT No.: PCT/EP11/59326

§ 371 Date: Dec. 10, 2012

(30) Foreign Application Priority Data
Jun. 11, 2010 (DE) 10 2010 017 333.9

(51) Int. Cl. A61M 15/00

(52) U.S. Cl. CPC A61M 15/0045
USPC 128/203.15

(57) ABSTRACT

The invention relates to a dispenser (1) for powdery compositions (M) contained in a separate pack, in particular compositions (M) contained in a blister pack (18), wherein the pack has a container part (14) with tear-off container cover (16), wherein furthermore an aspirated stream of air is sucked for the most part through the container part (14), after the container cover (16) has been removed from the container part (14), in such a way that two air streams (a, b) entering through slits (29) of a lid (19) empty the container part (15) from the direction of its two ends and, after coming together, pass into a swirl chamber (7) arranged upstream of a mouthpiece (3). In order to further optimize a dispenser in terms of flow technology, it is proposed that the air stream channel (27) designed as an arc-shaped path leads to the more distant end of the container part (14) and encompasses the container part (14) with an at least 180° curve area (52).

The invention relates to a dispenser for powdered compositions contained in a separate pack, in particular compositions contained in a blister pack, the pack having a container part with a pull-off container cover, furthermore an aspirated air stream being sucked predominantly through the container part, after the container cover has been removed from the container part, in such a way that two air streams entering through slits in a lid empty the container part from its two ends and, after combining, pass into a vortex chamber arranged upstream of a mouthpiece.

A dispenser of the type in question is known from WO 2009/138344 A1.

With regard to the known prior art, a technical problem of the invention is seen as that of further optimizing a dispenser of the type in question in terms of flow technology.

This problem is solved initially and primarily by the subject matter of Claim 1, this being based on the fact that the air stream channel configured as an arcuate path leads to the more distant end of the container part and surrounds the container part with an at least 180° curved region. As a result of this configuration, optimization in terms of flow technology is achieved, in particular with regard to the transport of the composition which is to be discharged out of the container part that forms a cavity and is to be inhaled. Thus furthermore, the discharge and transport of compositions which differ in particular with regard to grain size and/or mixture is improved as a result of the proposed solution. The air stream channel which is preferably connected to the ambient air via inlet openings in the lid, in particular slits, is connected in terms of air flow initially to the end of the receptacle of the dispenser that accommodates the container part in which the medium is stored, especially the end of said receptacle as viewed in the air flow direction and in particular more distant relative to the mouthpiece, for emptying out the composition from the container part, preferably as a result of the appropriate action of pressure and/or flow, into a portion of the air stream channel which is situated at the opposite end of the container part in the flow direction and adjoins the receptacle which accommodates the container part. Furthermore the portion of the air stream channel which is associated with the more distant end of the container part and is preferably connected via slits to the ambient air is guided so that it surrounds the container part or the receptacle which accommodates the container part, in particular with respect to a vertical projection of the air stream channel onto a housing base of the dispenser, more preferably with respect to a plane oriented perpendicular to the central axis of the vortex chamber, by at least 180°, so that the portion of the air stream channel guided as an arcuate path is also connected in terms of flow, in a combining region disposed upstream of the vortex chamber in the air flow direction, to the portion of the air stream channel adjoining the end opposite from the more distant end of the container part. Thus preferably starting from the air inlet region, that is to say starting from the slits preferably provided in the lid, a direct channel portion leading directly to the combining region and a container channel portion leading to the more distant end of the container part are produced, these two channel portions designated above, when combined, forming the air stream channel configured as an arcuate path, more preferably with a gradual transition of the two channel portions into one another, which is thus appropriately favorable in terms of flow technology, In this case, in particular the container channel portion of the air stream channel has a 180° curved region brought to the distant end of the container part. In this way, there is achieved a deflection, which is favorable in terms of flow technology, of the air flow which empties out the container part.

Further features of the invention are explained below, also in the description of the drawings, often in their preferred association with the subject matter of Claim 1 or with features of further claims. However, they may also be significant in an association with only individual features of Claim 1 or of the respective further claim or in each case independently.

Thus in a further preferred embodiment, it is provided that the cross-section of the arcuate path channel tapers in the direction of the combining. Accordingly in particular the portion of the arcuate path which opens into the combining region together with the portion of the air stream channel connected to the emptying end of the container part is reduced in its opening cross-section relative to the opening cross-section of the portion of the air stream channel preferably surrounding the container part by 180°, and also reduced relative to the free flow cross-section of the portion of the air stream channel which likewise opens into the combining region, that is to say also preferably by 20% to 70%, more preferably by 30% to 60%, further preferably by 40%, relative to the average cross-sectional dimension of the portion of the air stream channel which opens into the combining region or of the portion of the air stream channel having the 180° curved region. As a result of the cross-sectional tapering of the arcuate path channel in the direction of the combining portion, a nozzle-like mouth of the arcuate path channel is achieved in the combining region. During the through flow of air in the air stream channel which is achieved in the course of an inhalation, this leads to a negative pressure acting on the portion of the air stream channel extending from the container part and likewise opening into the combining region, so that accordingly the emptying out of the container part is assisted in an advantageous manner. Accordingly, in particular during inhalation, more particularly during the emptying out of the container part, a suction and pressure action on the composition stored in the container part is achieved in the air flow direction. Furthermore, the negative pressure produced as a result of the nozzle-like taper of the arcuate path channel at the end opposite from the more distant end of the container part, in particular at the beginning of the inhalation process, possibly leads to a type of initial action, and as a result of this negative pressure or suction action possibly at the outset, a part of the composition is discharged from the container part, after which a channel-like through-flow region is established in the region of the container part, for further blowing out of the composition via the stream of air guided through the rearward air stream channel.

It is also preferably provided that a narrow rib provided with concave side faces engages over the container part. This rib also preferably extends transversely to an air flow passing through the container part, furthermore the rib being designed in particular for the deflection of the air flow for entry into the opened container part and for discharge therefrom. In an advantageous manner, this air flow deflection takes place uniformly gently as a result of the concave side faces of the rib provided at the flow inlet and flow outlet. In a preferred configuration, as a result of the solution described above—with respect to a vertical section through the container part and the adjoining portions of the air stream channel—there results in particular a double S-shaped guiding of the air flow for passage through the opened container part and for emptying out the composition stored in the container part, preferably radius of the concavity of the rib being adapted to the cross-sectional radius of the channel portion to the rear of the container part or to the rear of the receptacle, furthermore preferably incorporating this radius.

In order in particular to achieve a first fixing of the container part to be inserted into the dispenser, in a development of the subject matter of the invention, it is provided that latching lugs engage over the container part, in particular a supporting portion of the container part, more preferably over the container cover which covers over the container part in the unused position. These latching lugs are preferably formed on the device, in particular flanking a region which accommodates the container part. In particular as a result of the configuration of the supporting portion of the blister pack surrounding the container part, which configuration is flexible in the usual way, the user can achieve easy insertion thereof by overcoming the projecting latching lugs. If in this case the latching lugs engage over the in particular tab-like, pull-off container cover, then in a further preferred configuration, the arrangement and configuration of the latching lugs is chosen so that the container cover can be pulled under the projecting ribs in particular as a result of rolling over the supporting portion on the container part, and then furthermore the latching lugs engage over the container part over the supporting portion thereof also in the pulled-off position of the container cover, thus also in particular in the use or inhalation position. Accordingly, preferably as a result of the latching lugs, the predefined orientation of the container part is also provided during inhalation.

Moreover air inlet slits in the lid region are preferred which extend above the 180° curved region of the air stream channel, so that accordingly during the inhaling process and accompanying suction through the air stream channel, ambient air flows in through the air inlet slits in the lid directly into the curved region of the air stream channel. The opening cross-section of the air inlet slits covering the air stream channel or the curved region thereof is preferably dimensioned in terms of the sum of the air inlet slits so that a sufficient air flow volume can be achieved for complete emptying of the container part during inhalation by the user.

In a further preferred configuration, the cavity for the container part is formed in an intermediate base of the dispenser preferably extending to the extent of the plane of the lid in the operating position, more preferably leaving an auxiliary air channel which is formed and extends below the intermediate base and which opens into the vortex chamber preferably outside the air stream channel that opens into the vortex chamber. Ambient air sucked in via the auxiliary air channel supports the uniform distribution of the composition introduced at the same time via the air stream channel into the vortex chamber, the air sucked in through the auxiliary air channel being combined with the composition-laden stream of air, within the vortex chamber.

In a further preferred configuration it is provided that the rib, which is also preferably provided with concave side faces, projects into the uncovered gap of the cavity, also preferably with the rib arranged at least approximately centrally, when viewed in the flow direction, with respect to the uncovered container part, so that also preferably at least approximately equal flow channel cross-sections are produced on the inflow and outflow side relative to the uncovered container part.

The invention is explained in greater detail below with reference to the appended drawings which only illustrate one exemplary embodiment. In the drawings:

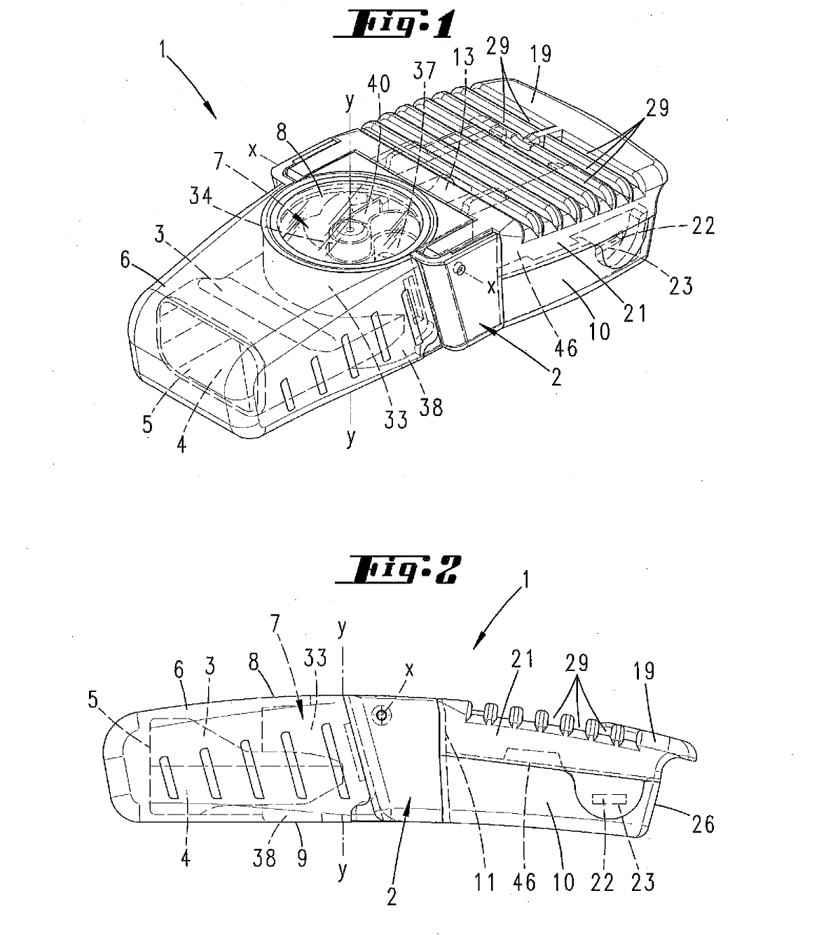

FIG. 1 shows a dispenser of the type in question in a perspective view, relating to the position when not in use;

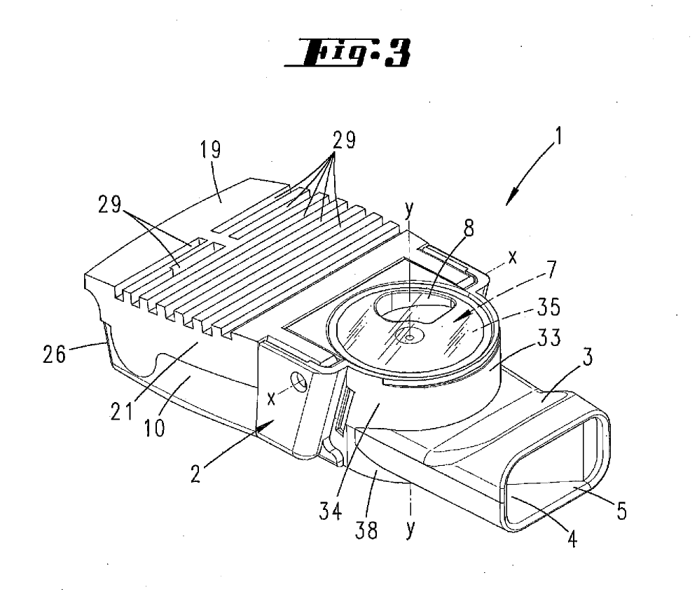

FIG. 2 shows the dispenser in a side view;

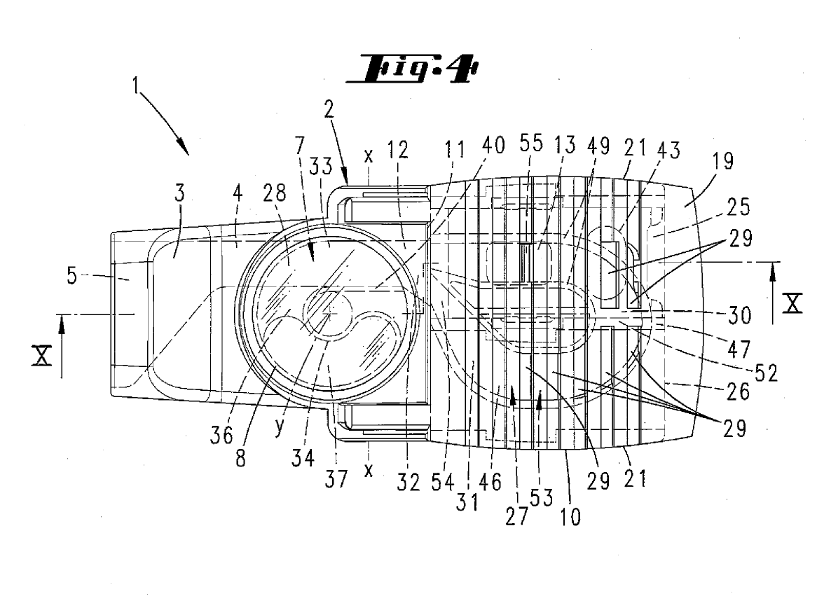

FIG. 3 shows the dispenser in a further perspective view, after removal of a protective cap for the suction mouth;

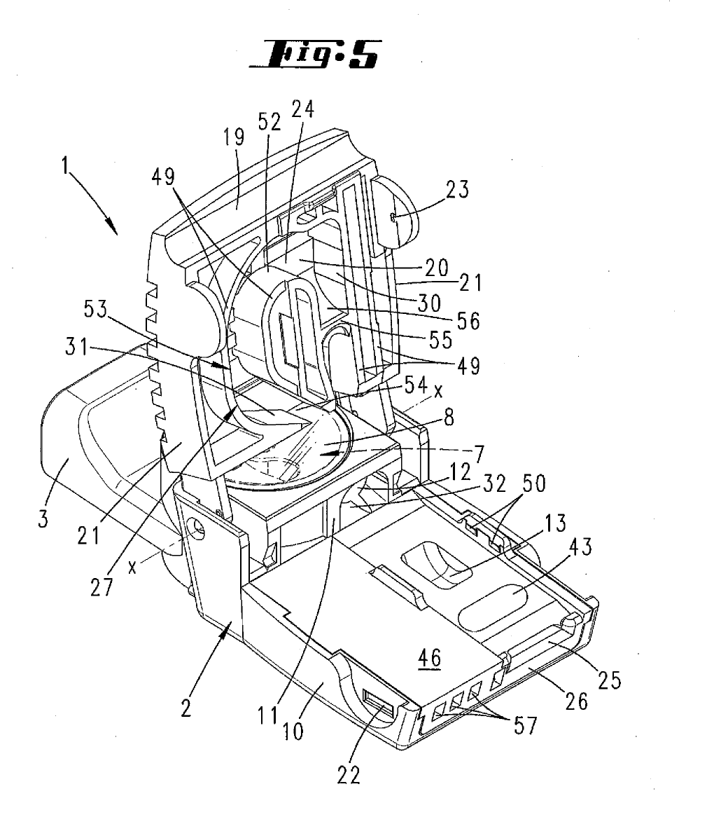

FIG. 4 shows a plan view in this connection;

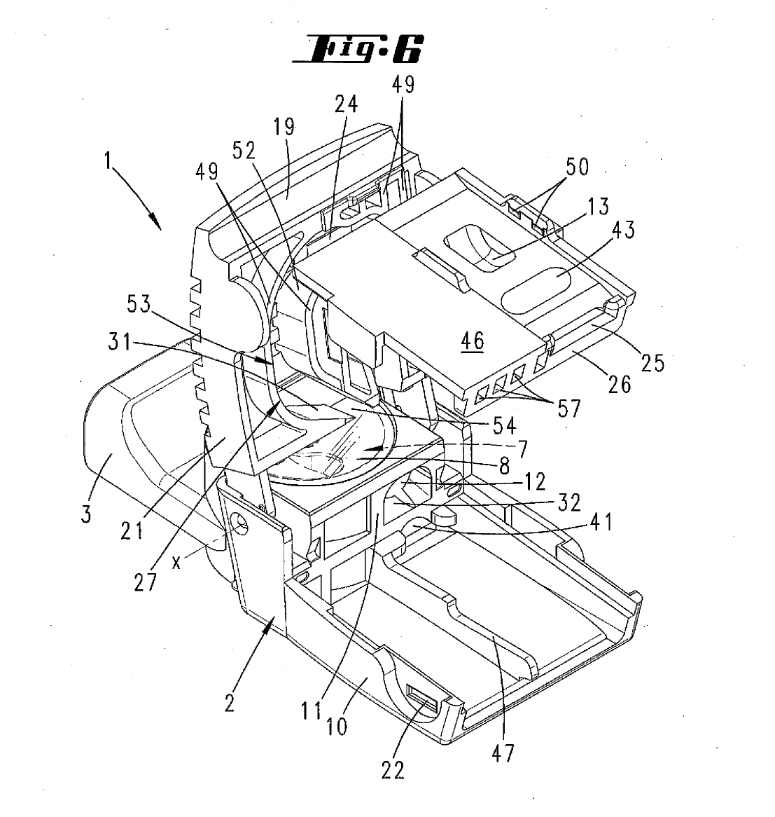

FIG. 5 shows the dispenser in a perspective view, relating to a standby position for fitting with a container part;

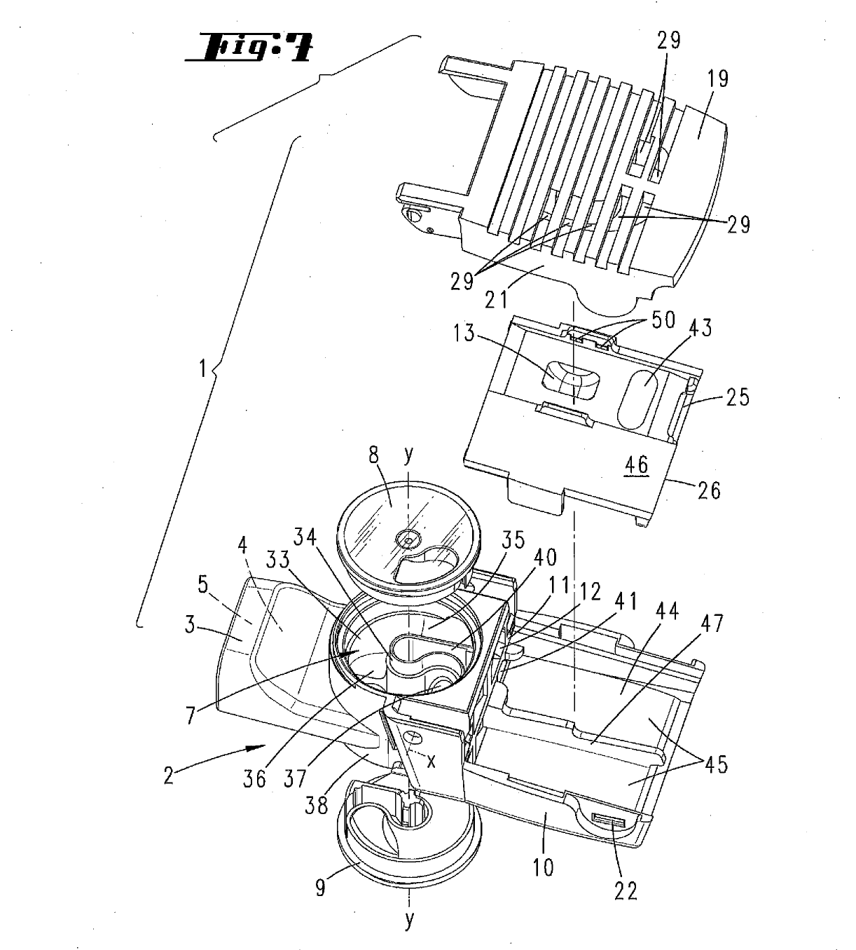

FIG. 6 shows a further perspective view of the dispenser in the fitting standby position, with an intermediate base shown in an exploded perspective view;

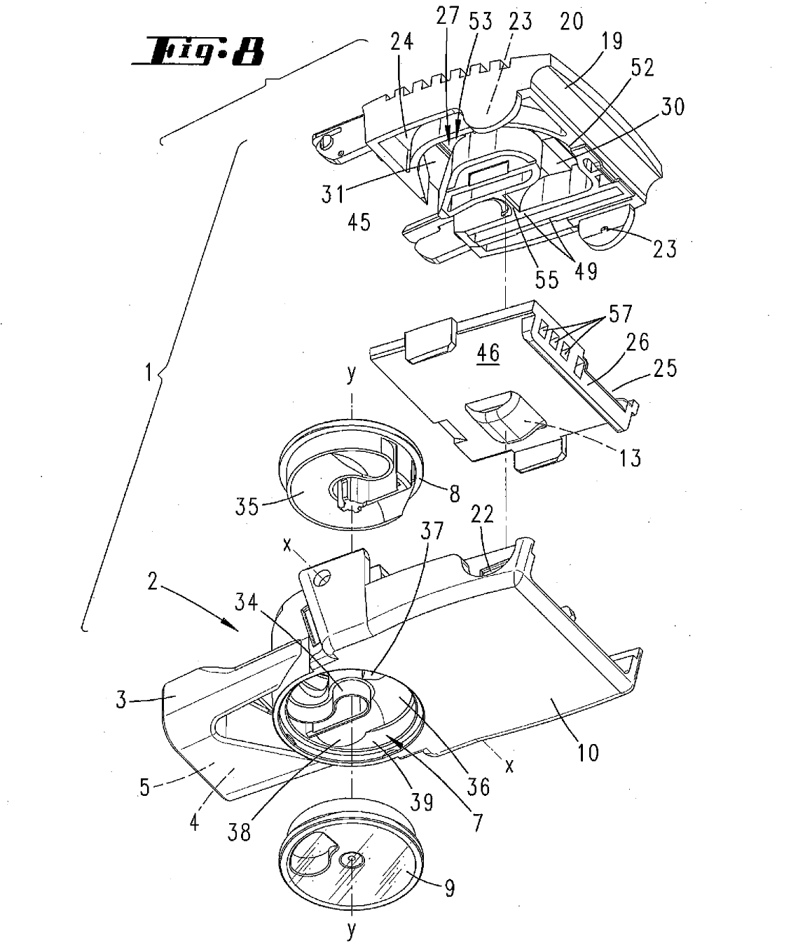

FIG. 7 shows the dispenser in an exploded perspective view;

FIG. 8 shows a further exploded perspective view of the dispenser;

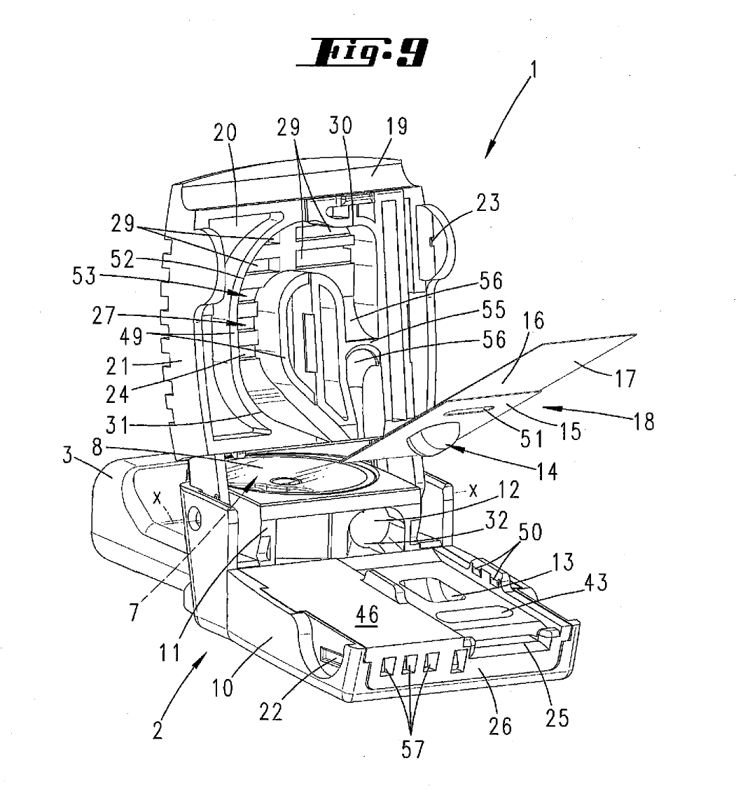

FIG. 9 shows a further perspective view of the dispenser in the fitting standby position in the course of fitting with a container part;

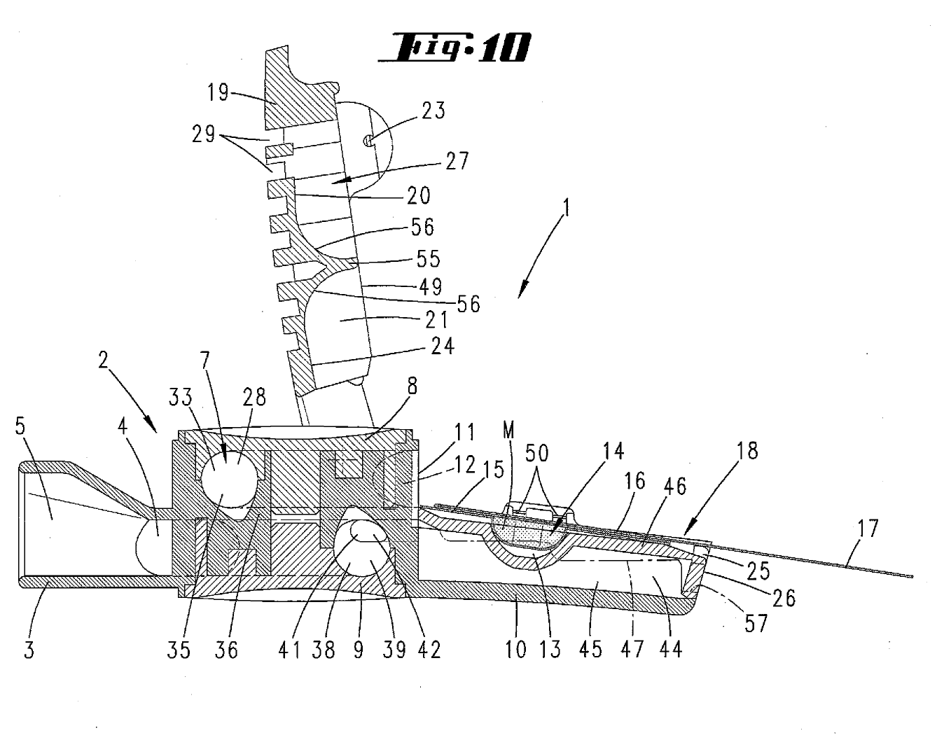

FIG. 10 shows the section along the line X-X in FIG. 4 with the container part inserted;

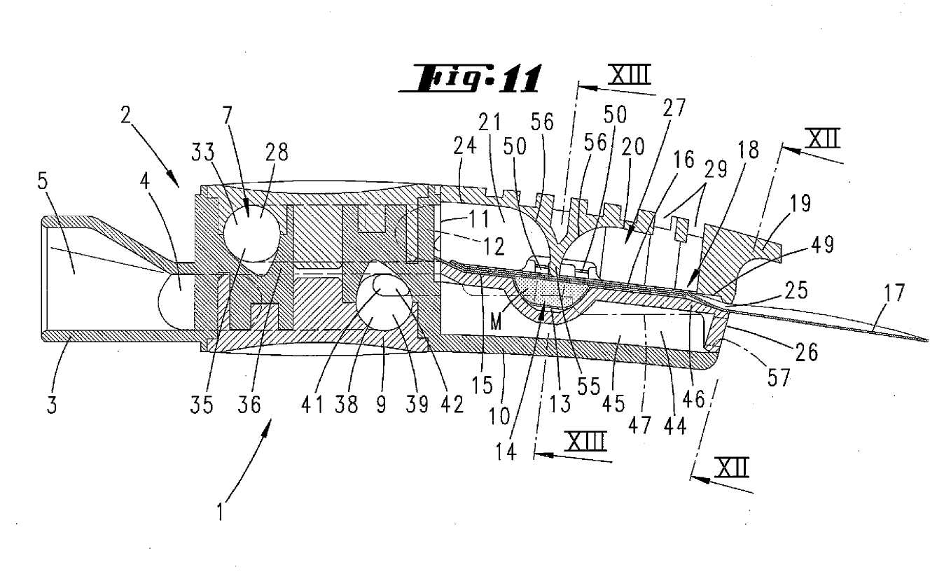

FIG. 11 shows a longitudinal sectional view corresponding to FIG. 10, but relating to the dispenser standby position;

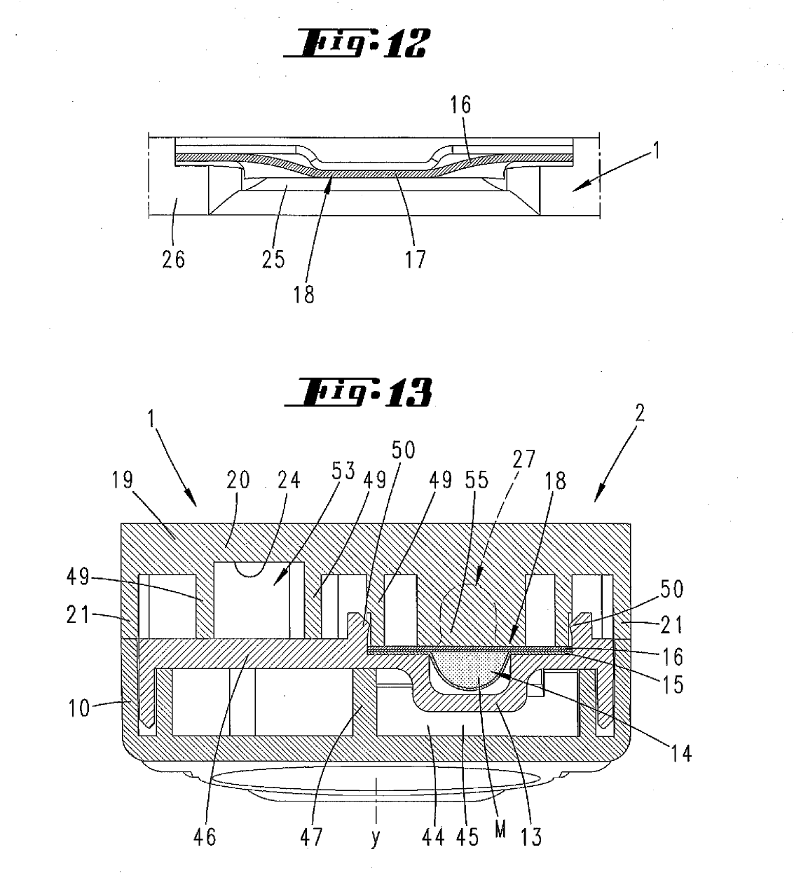

FIG. 12 shows the section along the line XII-XII in FIG. 11;

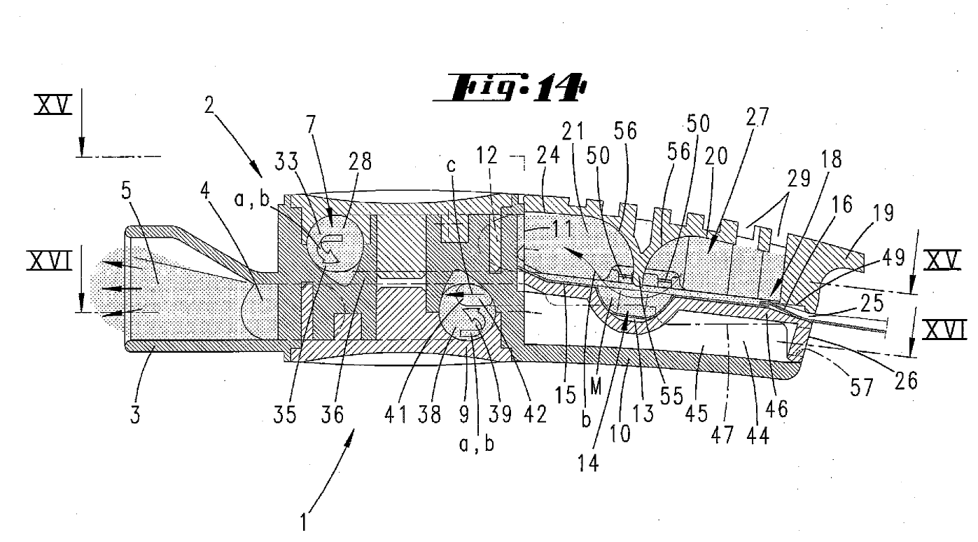

FIG. 13 shows the section along the line XIII-XIII in FIG. 11;

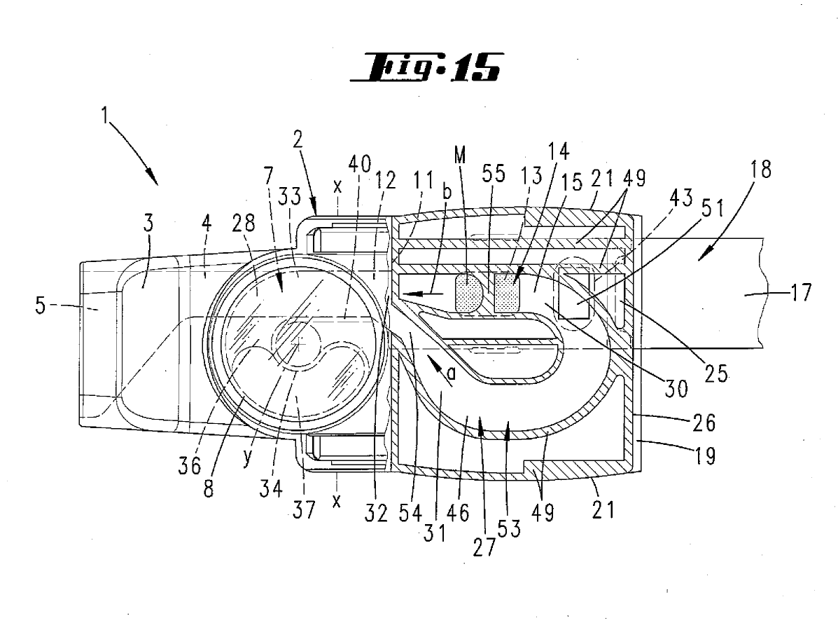

FIG. 14 shows a longitudinal sectional view corresponding to FIG. 11, but during an inhaling process;

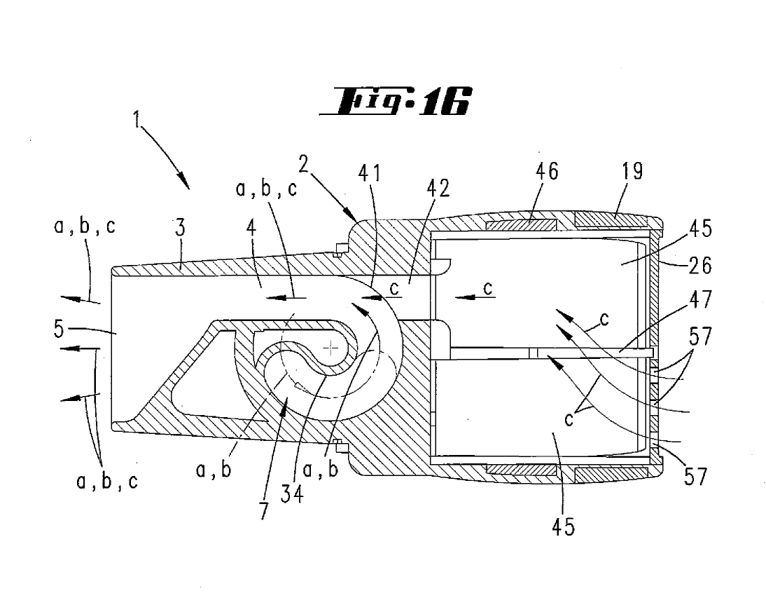

FIG. 15 shows the section along the section line XV-XV in FIG. 14;

FIG. 16 shows the section along the section line XVI-XVI in FIG. 14.

First of all with reference to FIG. 1, a dispenser 1 is illustrated and described in the form of an inhaler which is produced as a portable device that can be conveniently carried in the pocket. This has a substantially elongate rectangular housing 2, with a ratio of length to width of approximately 2:1 to 2.5:1 and a height when viewed perpendicular to the length which corresponds approximately to one quarter of the dimension of the length. The parts of the dispenser 1 are produced as injection molded plastics parts.

A mouthpiece 3 projects in prolongation of the longitudinal extent of the housing 2. With an overall one-piece configuration, the transition from the housing 2 to the mouthpiece 3 is waisted with respect to the width of the housing 2.

A suction channel 4 which terminates in a suction mouth 5 at the discharge end passes through the mouthpiece 3 in the longitudinal extent.

When the dispenser 1 is not in use, the mouthpiece 3 can be covered in accordance with the illustrations in FIGS. 1 and 2 by a closure cap 6. This latter takes up substantially the width of the housing 2 and can be plugged on in the region of the waist-like transition from the mouthpiece 3 to the housing 2.

In the housing 2, a circular vortex chamber 7 with a vortex chamber axis y extending substantially perpendicular to the suction channel 4 is associated with the transition region from the housing 2 to the mouthpiece 3. This vortex chamber 7 extends over two levels substantially over the entire height of the housing 2, the chamber cover 8 and the chamber base 9 being formed by cover plates shaped like circular discs which are preferably plugged onto the housing 2. In the illustrated embodiment, as also preferred, the chamber cover 8 and chamber base 9 are transparent.

The housing part 10 facing away from the mouthpiece 3 and adjoining the vortex chamber 7 is stepped in terms of height. The continuing surface plane of this housing part 10 which is vertically offset relative to the vortex chamber cover 8 extends approximately in a central plane with respect to the vertical extent of the housing 2. An air channel 12 aligned substantially in the longitudinal extent of the housing 2 opens on the end face, that is to say facing the offset plane of the housing part 10, in the step 11 which adjoins the vortex chamber 7 opposite from the mouthpiece 3. This air channel has a diameter which preferably corresponds approximately to 0.7 to 0.8 times the free step height. At the other end, this air channel 12 opens into the vortex chamber 7, in particular in the upper level of the vortex chamber 7.

The region of the housing part 10 which is stepped in height is initially configured to be open upwards, facing away from a housing part base, in this case substantially divided in the longitudinal extent by a vertical web 47 disposed centrally with respect to the width as viewed transversely to the longitudinal extent, so that accordingly two regions of at least approximately the same size are produced, one on each side of the vertical web 47. These regions are covered over by an intermediate base 46 which accordingly forms the offset surface plane of the housing part 10. The intermediate base 46 terminates substantially around the rim at the lower step edge, also below the mouth of the air channel 12 and at the boundary of the stepped region of the housing part 10, and is also preferably latched non-releasably to the housing part 10 in operation.

A receptacle 13 which opens towards the planar surface is formed in the intermediate base 46. This receptacle is slot-like in plan view and furthermore is shaped in the step 11 in axial extension of the air channel 12 at a distance from the mouth of the air channel. The contour and depth of the dish-like receptacle 13 is adapted to the contour and height of a container part 14 to be accommodated, appropriately chamfered in longitudinal extent at each end to accommodate a capsule-like container part.

As can be seen from the further illustrations, the container part 14 is configured in the manner of a blister pack, the container part 14 storing a powdered composition M. For this purpose the container part 14 is produced in the form of a dish from a plastics material, a planar supporting portion 15 also adjoining the peripheral opening rim of the container part 14. Overall the container part 14 is formed out of the supporting portion 15 as a recess.

In the position when not in use, the substance M stored in the container part 14 is sealed in by an aluminum foil which covers over the supporting portion 15 and the container part 14 over the entire surface area and constitutes the container cover 16. This container cover 16 can be pulled off from the supporting portion 15 in order to uncover the container part 14 and the substance M stored therein, for which purpose the aluminum foil or the container cover 16, starting from a narrow region of the supporting portion 15 oriented transversely to the longitudinal extent of the container part 14, extends freely in the opposite direction and covers over the region of the container cover 16 which seals the container part 14 and the supporting portion 15, resting freely on this region. The free end of the container cover 16 projects freely beyond the end peripheral edge of the supporting portion 15 which is directed away from the bent-around region of the container cover 16, in order to form a tab-like pull-off handgrip 17.

The receptacle 13 formed in the intermediate base 46 for the container part 14 is positioned in a region which is offset at a lower vertical level than the surface of the intermediate base. The vertical offset corresponds substantially to the material thickness of the blister 18 made up of the container part 14, container cover 16 and supporting portion 15 outside the container part 14. Furthermore in particular the length of the recess, as viewed along the longitudinal extent of the housing 2, is adapted to the length of the supporting portion 15, as viewed along the longitudinal extent of the container part 14. The surface of the recess which surrounds the receptacle 13 serves as a support for the supporting portion 15 when the blister 18 is inserted.

A lid 19 is associated with the housing part 10. This lid is articulated on the housing part 10 in the region of the step 11 in such a way that it can be pivoted about an axis x aligned transversely to the longitudinal extent of the housing 2. The lid 19 has a top 20. Side walls 21 extending on both sides along the longitudinal extent of the housing 2 are formed in one piece with the lid top 20 and, in the closed position of the lid, flank the associated side surfaces of the fixed housing part 10.

The lid 19 is latched in the closed position of the lid, for which purpose latching lugs 23 are provided on the side walls 21 of the lid 19 and penetrate into latching recesses 22 on the housing part. The latching thus produced can easily be overcome by the user in order to open the lid 19.

In the closed position of the lid, the underside 24 of the top rests, by means of vertical webs 49 formed thereon, on the facing upper side of the intermediate base 46, that is to say preferably on the planar surface surrounding the recess which accommodates the receptacle 13. With the blister 18 inserted into the receptacle 13 in the intermediate base and, at the same time, with the vertical offset in the intermediate base 46 being compensated, the underside 24 of the top or the vertical webs 49 provided on the underside of the top preferably rests on the blister 18, in particular on the container cover 16 which forms the pull-off handgrip 17 in the free end region. Accordingly, the inserted blister 18 is gripped in the closed position of the lid, on the one hand, by the container part 14 being fitted in an approximately positively locking manner in the receptacle 13 and, on the other hand, on the upper side and underside, by the lid top 20 and the associated surface of the recess in the intermediate base, furthermore lateral support of the blister 18 via the supporting portion 15 being provided, on the one hand, by the step 11, and, on the other hand, by the peripheral boundary of the recess.

Moreover, by means of the vertical webs 49 provided on the underside of the lid 19, the lid 19 is also supported on the neighboring planar surface of the intermediate base 46 which is raised relative to the recess which accommodates the blister 18, at the same time the vertical webs 49 of the lid 19 at least partially delimiting flow paths.

In the longitudinal extent of the recess accommodating the blister 18 in the intermediate base 46, latching lugs 50 are formed on both sides on the edge of the recess and, in the fitted position of the container part, engage over the supporting portion 15 and, in the position when not in use, also engage over the container cover 16, so that a blister 18 inserted when the lid 19 is open is trapped as a result of the engagement of the latching lugs 50 in the fitted position. As a result of the thin-walled and optionally resilient configuration in particular of the supporting portion 15 of the blister part, the latching lugs 50 can be overcome during insertion of the blister 18 and also during removal.

Furthermore a marking 43 is applied, in particular in the form of a color marking, on the surface of the recess in the intermediate base at the outlet of the receptacle 13. In the fitted position of the blister 18, this marking 43 is vertically aligned with a window-like cutout 51 in the supporting portion 15, which window-like cutout 51 is uncovered during pulling off the container cover 16 for preparation for inhalation.

The blister 18 should be inserted into the dispenser 1 in such a way that the wrapped-around edge of the container cover 16 is aligned parallel to the lid axis x and disposed facing the air channel 12, and furthermore the freely protruding pull-off handgrip 17 projects in the opposite direction beyond the edge of the recess out of the housing 2, passing through a slit-like pull-off opening 25 appropriately provided between the intermediate base 46 and the lid 19 when this is pivoted into the closure position. The width of the pull-off opening when viewed transversely to the longitudinal extent of the housing 2 is adapted to the width of the container cover 16. The vertical height of the pull-off opening 25 corresponds substantially to the material thickness of the container cover 16.

A correct orientation of the blister 18 to be inserted in the dispenser 1 is ensured by an eccentric arrangement of the receptacle 13 in the longitudinal extent of the dispenser 1 or in the recess in the intermediate base 46. Accordingly the blister 18 has along the longitudinal extent different leg lengths of the supporting portion 15 adapted to the eccentric arrangement of the receptacle 13, so that the blister 18 can be inserted exclusively in the predetermined orientation.

In the closed position of the lid, the vertical webs 49 projecting on the underside of the lid together with the facing surface of the intermediate base 46 form an air stream channel 27 which is guided in the manner of an arcuate path with respect to a vertical projection onto the intermediate base 46. The air stream channel 27 initially leads as container channel portion 30 to the end of the receptacle 13 in the intermediate base or of the container part 14 accommodated in the receptacle 13, which end is more distant with respect to the air channel 12 in the housing. Starting from this end, the air stream channel 27 extends, forming a 180° curved region 52, to a channel portion extending at least approximately parallel to the longitudinal orientation of the receptacle 13, said channel portion surrounding the receptacle 13 on one side and merging into a direct channel portion 31.

At the other end of the receptacle 13, in a substantially axial extension of the air channel 12 in the housing, the air stream channel 27 is also formed for direct connection in terms of flow to the air channel 12 in the housing in the closed position of the lid. The direct channel portion 31 opens together with the portion of the air stream channel 27 associated with the air channel 12 into a combining portion 32, which in the illustrated exemplary embodiment is formed in the region of the opening in the air channel 12 in the housing facing the air stream channel 27.

The arcuate path channel 53 formed in particular from the container channel portion 30 and the direct channel portion 31 is tapered in the direction towards the combining portion 32 with regard to its cross-sectional area through which the flow can pass freely relative to the average cross-sectional area of the air stream channel 27, preferably tapered by approximately 40% of the average cross-sectional area. This tapered, nozzle-like end portion of the direct channel portion 31 is provided with the reference 54.

Furthermore a rib 55 is formed on the underside of the lid, to overlie the receptacle 13 or the container part 14 inserted in the receptacle 13. This rib extends transversely between two vertical webs 49 which engage over the receptacle 13 and delimit a portion of the air stream channel 27, and in this case the rib has a height when viewed in the closed position of the lid which is chosen so that in the inhalation standby position, the downwardly projecting free end peripheral edge of the rib 55 projects into in the gap above the opened container part 14 which is uncovered after the container cover 16 has been pulled off.

The rib 55 offers a deflection of the air stream channel 27 in the direction towards the receptacle 13 or towards the container part 14 located in the receptacle 13 and also a deflection out of this latter. For at least low-loss deflection, with respect to a vertical section according to FIG. 11, the rib 55 is provided on both sides with concave side faces 56. These are provided with a radius preferably adapted to the cross-sectional radius of the air stream channel 27, furthermore the rib 55 having, in the free end region thereof pointing downwards in the direction towards the receptacle 13, a width which, when viewed in the flow direction, corresponds approximately to 0.1- to 0.3 times, preferably approximately 0.2 times, the length of the receptacle 13 when viewed in the same direction.

Furthermore, with respect to a longitudinal extent of the receptacle 13 or of the container part 14, the rib 55 is disposed so as to be oriented centrally and transversely to the longitudinal extent.

Accordingly, after the container cover 16 has been pulled off, according to FIG. 14, as a result of the arrangement and configuration of the rib 55 described above, the air stream channel 27 is directed substantially in the shape of a double S, passing through the container part 14.

Furthermore, air inlet slits 29 are provided in the lid top 20 overlapping the arcuate path channel 53, also in particular overlapping the curved region 52, these air inlet slits being preferably open upwards in the closed position of the lid and also preferably open laterally, passing through the associated side wall 21. Thus a top like an inlet grid is achieved in particular for the curved region 52.

The air channel 12 in the housing opens towards the housing part 10, and at the other end opens into a deflecting portion 28 of the vortex chamber 7, and also in the upper level 33 of the vortex chamber 7, this upper level 33 extending substantially above the separating plane between the intermediate base 46 and the lid 19.

The upper level 33 of the vortex chamber 7 is substantially annular, for which purpose an annular wall 34 which extends over the entire height of the upper level 33 is provided. Starting from the air channel inlet, the annular channel 35 of the upper level 33 extends approximately over an angle of 270° (anti-clockwise in relation to the illustration in FIG. 4) before the annular space 35, passing through an aperture 37 which opens the base 36 of the level, merges into an annular channel 39 similarly formed in the level 38 disposed underneath. This annular channel 39 is also delimited radially on the inside by the annular wall 34 which runs through substantially over the entire vertical height of the vortex chamber 7.

The opening 37 in the base is followed immediately downstream, in the region of the upper level 33, by a partition wall 40 which closes the annular space in the circumferential flow direction in relation to the inlet of the air channel 12 and extends radially from the associated annular wall portion.

The annular channel 39 of the lower level 38 extends from the 90° curved region of the upper annular channel 35 over approximately 270° as far as the connection to the suction channel 4 of the mouthpiece 3.

The aperture 37 which connects the levels or the annular channels 35 and 39 extends over an angular range of approximately 180°, which when viewed in the flow direction covers the angular range from 90° to 270° of the upper annular channel 35 and the angular range from 270° to 90° of the lower annular channel 39. Accordingly a transition for the two annular channels is provided over a range of approximately 180°.

Thus a substantially helical guidance of air is produced in the vortex chamber 7, with the air which is sucked through the vortex chamber 7 circulating by a total of 360°, also including passage through two levels disposed below one other.

In the annular space 39 of the lower level 38 of the vortex chamber 7, outside air opening 41 opens in vertical alignment with the mouth of the air channel 12 into the upper level 33, and the outside air channel 42 of said outside air opening passes through the housing 2 in such a way that the outside air opening 41 opens tangentially into the annular space 39. At the other end, the outside air channel 42 extends along the longitudinal extent of the housing 2 and opens at the underside of the opening of the air channel 12 in the step into the space 44 left between the intermediate base 46 and the housing base. Said intermediate space forms an auxiliary air channel 45 which is formed on either side of the vertical web 47 in the intermediate base. The region of the intermediate space 44 formed below the receptacle 13 is closed to the rear of the housing by a vertically extending rear wall 26 of the intermediate base 46. In this end portion associated with the rear wall 26, the vertical web 47 which substantially divides the longitudinal extent of the intermediate space 44 is reduced in height, in order for intermediate base regions formed adjacent to one another to be connected in terms of flow, outside air entry openings 57 in the rear wall 26 being associated with the region of the intermediate space formed underneath the arcuate path channel 53. Accordingly, in the inhalation operating position, these outside air inlet openings 57 are adjacent to the container cover 16 projecting out of the housing 2 at the rear wall.

For inhalation of a composition M, first of all, after the lid 19 has been pivoted up into the open position according to FIG. 9, the blister 18 is inserted in such a way that the container part 14 is accommodated in the receptacle 13, with the supporting portion 15 of the blister being supported on the associated recessed surface of the intermediate base 46. In this ease, the latching lugs 50 which are also provided overlie the supporting portion 15 and preferably the container cover 16 which is situated in the closure position and which also, as a result of the guidance at the fold in the end region of the blister 18, is double-layered in particular in the region of the latching lugs 50. The freely projecting pull-off handgrip 17 protrudes freely beyond the end of the housing for operation.

After closure of the lid 19, the blister 18 is secured in the housing 2. The container part 14 is then opened by the container cover 16 being pulled off by unrolling, for which purpose the pull-off handgrip 17 which projects through the pull-off opening 25 is pulled in the pull-off direction r. This pull-off operation is visible as a result of a transparent construction of the lid 19, in particular a transparent construction of the portion of the lid 19 overlapping the displacement region of the container cover 16. The marking 43 provided on the surface of the intermediate base 46 serves the user as a guide to the position up to which the container cover 16 must be pulled, in order to ensure that the composition M stored in the container part 14 is completely uncovered. The window-like cutout 51 in the supporting portion 15 which is initially covered over by the container cover 60 is uncovered during the unrolling action of pulling off the container cover 16, so that the marking 43 is then visible through the cutout 51. Accordingly this provides guidance for the user for proper use of the dispenser 1.

After the container cover has been pulled off from the container part 14, the composition M is exposed in the flow path. Due to the absence of a cover, the container part 14 is now part of the flow path, and accordingly is connected in terms of flow, at one end, to the portion of the air stream channel 27 leading to the combining portion 32 and also to the air channel 12, and, at the other end, to the portion of the air stream channel 27 which is like an arcuate path channel and which in turn opens at the end into the combining portion 32.

For inhalation, the dispenser 1 is held like a whistle, preferably between the thumb and forefinger. By sucking in via the mouthpiece 3, air enters into the arcuate path channel 53 of the air stream channel 27 through the inlet slits 29, and then the incoming air is divided for passage through the direct channel portion 31 leading directly to the combining portion 32 and for passage through the container channel portion 30 leading directly to the container part 14. The powdered composition M stored in the container part 14 is discharged via this container channel portion 30, and then the fraction of air b mixed with the composition is combined in the combining portion 32 with the fraction of air a through the direct channel portion 31. As a result of the cross-sectional configuration of the direct channel portion 31 which tapers towards the combining portion 32, there is achieved, on the portion of the air stream channel 27 which likewise opens into the combining portion 32, a suction effect which assists the emptying out of the container part 14 due to the blown air introduced via the container channel portion 30. Passage through the helical, double-level vortex chamber 7 produces uniform distribution of the substance M in the air stream. Before exiting from the vortex chamber 7 into the suction channel 4 of the mouthpiece 3, further outside air c enters via the outside air opening 41. The outside air c which is sucked in here as auxiliary air passes through the outside air channel 42 into the vortex chamber 7, the outside air opening 41 which opens into the lower level 38 being oriented in the direction of the opening opposite from the suction channel 4. The outside air stream c is combined with the composition-laden air stream a, b in the lower level 33 of the vortex chamber 7 immediately upstream of the transition into the suction channel 4.

During the inhaling process, the free container-covering tab closes at least the majority of the pull-off opening 25, this counteracting the intake of auxiliary air.

As a result of the configuration described, air is drawn in and/or guided within the housing 2 while bypassing the container cover 16, which is displaced into the pull-off position for inhalation. The suction air stream which empties out the composition M from the container part 14 is guided without interference. As a result of the guidance of the air stream channel 27 in the manner of an arcuate path channel, in particular also as a result of the nozzle-like taper of the direct channel portion 31 in the direction of the combining portion 32, the guidance of air, in particular of the composition-laden stream of air, is optimized. The dispenser 1 is suitable for inhalation of different compositions M, in particular with regard to compositions which differ with regard to grain size and/or mixture.

All features disclosed are (per se) essential to the invention. The entire content of the disclosure of the associated/appended priority documents (copy of the prior application) is hereby incorporated into the disclosure of the application, for the purpose of also including features of these documents in claims of the present application. The subsidiary claims characterize in their optional sequence of dependency independent inventive modifications of the prior art, in particular in order to file divisional applications on the basis of these claims.

### LIST OF REFERENCES

1 dispenser
2 housing
3 mouthpiece
4 suction channel
5 suction mouth
6 closure cap
7 vortex chamber
8 chamber cover
9 chamber base
10 housing part
11 step
12 air channel
13 receptacle
14 container part
15 supporting portion
16 container cover
17 pull-off handgrip
18 blister
19 lid
20 top
21 side wall
22 latching recess
23 latching projection
24 underside of the top
25 pull-off opening
26 rear wall
27 air stream channel
28 deflecting portion
29 air inlet slit
30 container channel portion
31 direct channel portion
32 combining portion
33 level
34 annular wall
35 annular channel
36 base of the level
37 aperture
38 level
39 annular channel
40 partition
41 outside air opening
42 outside air channel
43 marking
44 intermediate space
45 auxiliary air channel
46 intermediate base
47 vertical web
48 - -
49 vertical web
50 latching lug
51 cutout
52 curved region
53 arcuate path channel
54 tapered portion
55 rib
56 concave surface
57 outside air entry opening
a stream of air
b stream of air
c stream of air
r pull-off direction
x axis of the lid
y vortex chamber axis
M composition

1. Dispenser (1) for powdered compositions (M) contained in a separate pack, in particular compositions contained in a blister pack (18), the pack having a container part (14) with a pull-off container cover (16), furthermore an aspirated air stream being sucked predominantly through the container part (14), after the container cover (16) has been removed from the container part (14), in such a way that two air streams (a, b) entering through slits (29) in a lid (19) empty the container part (15) from its two ends and, after combining, pass into a vortex chamber (7) arranged upstream of a mouthpiece (3), wherein the air stream channel (27) configured as an arcuate path leads to the more distant end of the container part (14) and surrounds the container part (14) with an at least 180° curved region (52).

2. Dispenser according to claim 1, wherein the cross-section of the arcuate path channel (53) tapers in the direction of combining.

3. Dispenser as claimed in claim 1, wherein a narrow rib (55) provided with concave side faces (56) engages over the container part (14).

4. Dispenser as claimed in claim 1, wherein latching lugs (50) engage over the container cover (16).

5. Dispenser as claimed in claim 1, comprising air inlet slits (29) in the lid region which extend above the 180° curved region (52).

6. Dispenser as claimed in claim 1, comprising an auxiliary air channel (45) which extends below an intermediate base (46) forming the cavity for the container part (14) and opens into the vortex chamber (7).

7. Dispenser as claimed in claim 1, wherein the rib (55) projects into the uncovered gap of the cavity.
